(19) United States
(12) Patent Application Publication
(10) Pub. No.: US 2024/0312628 A1
(43) Pub. Date: Sep. 19, 2024

(54) LEARNING DEVICE, DETERMINATION DEVICE, METHOD FOR GENERATING TRAINED MODEL, AND RECORDING MEDIUM

(71) Applicant: NEC Corporation, Minato-ku, Tokyo (JP)

(72) Inventor: Yuji OHNO, Tokyo (JP)

(73) Assignee: NEC Corporation, Minato-ku, Tokyo (JP)

(21) Appl. No.: 18/273,455

(22) PCT Filed: Mar. 29, 2021
(86) PCT No.: PCT/JP2021/013208

§ 371 Date: Jul. 20, 2023

(51) Int. Cl. G16H 50/20

(52) U.S. Cl. CPC G16H 50/20

(57) ABSTRACT

A learning device of the present invention is provided with: an acquiring means for acquiring biometric information of a patient who may possibly become in agitation, and biometric information of a non-patient; and a model generating means for using the biometric information of the patient and the biometric information of the non-patient to generate an agitation determination model for determining whether, on the basis of the biometric information of a subject patient, the subject patient has become in agitation or has not become in agitation.

### TECHNICAL FIELD

The present invention relates to a learning device, a determination device, a method for generating a trained model, and a recording medium.

### BACKGROUND ART

In a medical or care field, a patient may possibly become in agitation. If the patient becomes in agitation, the risk of removing or pulling a tube, a needle and the like, tumbling, falling down, or the like increases, and the patient may be injured. Therefore, a technique for detecting such a patient's agitated state in advance is known.

PTL 1 discloses a biometric information processing system that determines identification information indicating whether a condition of a target patient has changed as compared with a normal state based on a feature amount of input biometric information of the target patient, and estimates coping information for the target patient based on the identification information and a coping prediction parameter trained in advance.

### CITATION LIST

### Patent Literature

PTL 1: WO 2019/073927 A

### SUMMARY OF INVENTION

### Technical Problem

It is necessary to accurately determine whether the patient has become in agitation in order to reduce the risk of the patient's removing or pulling a tube, a needle, and the like, tumbling, falling down, and the like. In order to accurately determine whether the patient is in agitation, it is preferable to increase the accuracy of the model for determining the agitated state disclosed in PTL 1.

Therefore, the present invention has been made to solve the above problem, and an object thereof is to provide a device and the like capable of improving the accuracy of a model for determining a patient state.

### Solution to Problem

A learning device according to an aspect of the present invention is provided with an acquisition means that acquires patient biometric information and non-patient biometric information, the patient being a person who may possibly become in agitation, and a model generation means that generates an agitation determination model that determines whether a target patient has become in agitation or has not become in agitation based on biometric information of the target patient by using the patient biometric information and the non-patient biometric information.

A determination device according to an aspect of the present invention is provided with a determination means that determines whether the target patient has become in agitation using the biometric information of the target patient and the agitation determination model in which the agitation determination model is a trained model generated by a learning device including an acquisition means that acquires patient biometric information and non-patient biometric information, the patient being a person who may possibly become in agitation, and a model generation means that generates an agitation determination model that determines whether a target patient has become in agitation or has not become in agitation based on biometric information of the target patient by using the patient biometric information and the non-patient biometric information.

A method for generating a trained model according to an aspect of the present invention causes a computer to perform a process including acquiring patient biometric information and non-patient biometric information, the patient being a person who may become in agitation, and generating an agitation determination model for determining whether a target patient has become in agitation or has not become in agitation based on biometric information of the target patient by using the patient biometric information and the non-patient biometric information.

A recording medium according to an aspect of the present invention stores a program that causes a computer to perform a process including acquiring patient biometric information and non-patient biometric information, the patient being a person who may become in agitation, and generating an agitation determination model for determining whether a target patient has become in agitation or has not become in agitation based on biometric information of the target patient by using the patient biometric information and the non-patient biometric information.

### Advantageous Effects of Invention

According to the present invention, the accuracy of the model for determining the state of the patient is improved.

### BRIEF DESCRIPTION OF DRAWINGS

FIG. 1 is a block diagram illustrating a configuration of a learning device 10 according to a first example embodiment.

FIG. 2 is a flowchart illustrating a flow of an operation performed by the learning device 10 according to the first example embodiment.

FIG. 3 is a block diagram illustrating a configuration of an agitation determination system 200 according to a second example embodiment.

FIG. 4 is a flowchart illustrating a flow of an operation performed by the agitation determination system 200 according to the second example embodiment.

FIG. 5 is a block diagram illustrating an example of a hardware configuration.

### EXAMPLE EMBODIMENT

Hereinafter, each example embodiment of the present invention will be described with reference to the drawings.

In each example embodiment of the present invention, an agitation determination model is a trained model that determines whether a patient has become in agitation. The agitated state indicates a state in which the patient is uneasy. The agitated state may include a state in which his/her mind cannot be normally controlled. In addition, the agitated state may include a state caused by delirium of the patient. The agitated state may be caused by a mental or physical factor of the patient. It has been found that a patient often takes a problem behavior when being in agitation. In other words, a patient in an agitated state is highly likely to take a problem behavior. Therefore, by grasping whether the patient has become in agitation, it is possible to predict whether the patient is likely to cause a problem behavior. Here, the problem behavior of the patient is, for example, behavior that requires some measure by a medical worker who performs a medical treatment on the patient in response to the behavior. The problem behavior of the patient is, for example, leaving the bed, wandering alone, loitering, going to another floor in the hospital, removing the fence of the bed, falling from the bed, touching a drip or tubing, removing a drip or tubing, making a strange sound, making violent speech, acting violently, or the like. Whether the behavior of the patient is relevant to the problem behavior may be determined according to the condition of the patient. Here, the condition of the patient includes at least one of a cognitive function, a physical function, and a motor function. In each example embodiment of the present invention, the agitation determination model may determine whether the patient is causing a problem behavior. Hereinafter, a normal state of the patient, that is, a state not being in agitation is referred to as a non-agitated state.

In each example embodiment of the present invention, a patient is a person who takes a medical treatment by a medical worker. The patient may be a person who may possibly become in agitation. That is, the patient may be a person whose chance of becoming in agitation is a predetermined probability or more. In addition, for example, there is a high possibility that a person who has developed a specific disease, is taking a specific drug, has a reduced cognitive function, has blood loss, has a pain in the body, or the like, who satisfies at least one of the characteristics, will become in agitation. The patient may be a person who applies at least one of the features described above. Furthermore, the patient may include at least one of a hospitalized patient, a discharged patient, an outpatient, and the like. The patient is not limited to the above as long as the patient is a subject to the agitated state determination.

In each example embodiment of the present invention, the non-patient is, for example, a healthy person. The healthy person is, for example, a person who can perform daily living activities by himself/herself, has no underlying disease, does not need assistance or care of another person, or the like. The non-patient may be a person who is less likely to become in agitation. In other word the non-patient may be a person whose chance of becoming in agitation is equal to or less than a predetermined probability. In addition, the non-patient may be a person who does not have the feature of the person who is likely to cause the above-described agitated state. A person whose chance of becoming in agitation is a probability equal to or more than the first threshold value may be defined as a patient, and a person whose chance of becoming in agitation is a probability equal to or less than the second threshold value smaller than the first threshold value may be defined as a non-patient.

In the example embodiments of the present invention, the biometric information is information that changes with the life activity of a person. That is, the biometric information is time-series information indicating a change associated with human life activity. The biometric information is, for example, at least one of a heart rate, heart rate variability, a respiratory rate, a blood pressure value, a body temperature, a skin temperature, a blood flow rate, a blood oxygen saturation, a body motion, and the like. The biometric information may include other information used for agitated state determination. The biometric information is measured using, for example, at least one sensor worn by the measured subject. The measured subject includes a patient and a non-patient. The sensor is, for example, a heart rate sensor, a respiration rate sensor, a blood pressure sensor, a body temperature sensor, a blood oxygen saturation sensor, an acceleration sensor, or the like. The measured subject may wear a device on which one sensor is mounted or a device on which a plurality of sensors is mounted. The measured subject may wear a plurality of devices. The device is mainly a wearable device, and specific examples thereof include a smart watch, a smart band, an active tracker, a clothing sensor, a wearable heart rate sensor, and the like. Furthermore, the biometric information may be extracted from, for example, image information acquired by an imaging device (a camera or the like) installed in a room of the measured subject, voice of the measured subject, and sound information in a surrounding environment of the measured subject. The room of the measured subject is, for example, a hospital room or the like.

### First Example Embodiment

A configuration of a learning device 10 according to a first example embodiment will be described. The learning device 10 according to the present example embodiment generates an agitation determination model.

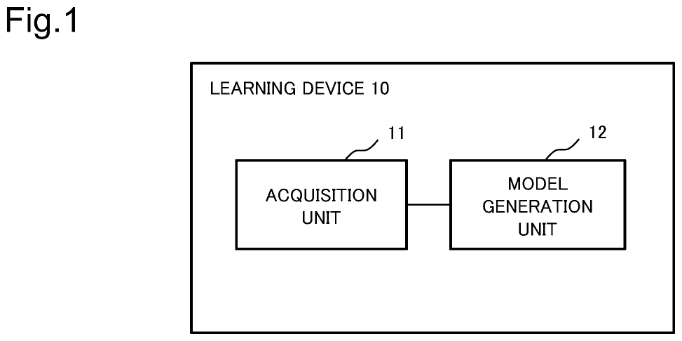

FIG. 1 is a block diagram illustrating a configuration of the learning device 10 according to the present example embodiment. The learning device 10 illustrated in FIG. 1 includes an acquisition unit 11 and a model generation unit 12.

The acquisition unit 11 is an acquisition means that acquires patient biometric information and non-patient biometric information. In other words, the acquisition unit 11 acquires biometric information of a patient who is a person who may possibly become in agitation and biometric information of a non-patient.

As an example, the biometric information is stored in a storage device (not illustrated) or the like in association with a measured subject ID for identifying a measured subject in association with time information indicating a time when the biometric information is measured. The acquisition unit 11 may acquire the biometric information of the measured subject from the storage device.

The acquisition unit 11 may acquire the biometric information of the measured subject associated with the time information from a sensor or a device communicably connected to the learning device 10 via a wireless or wired communication network. Furthermore, the acquisition unit 11 may acquire one or both of the patient biometric information and the non-patient biometric information at a predetermined timing.

Note that the acquisition unit 11 may acquire state information indicating the state of the measured subject at the time when the biometric information is measured. When the measured subject is a patient, the state is, for example, an agitated state or a non-agitated state. When the measured subject is a non-patient, the state is, for example, a resting state and a non-resting state which is a state other than the resting state. The resting state will be described later. The state information may be acquired from a storage device, a sensor, or a device similarly to the biometric information described above. Furthermore, the state information may be, for example, medical record information indicating information described in a medical document of the measured subject. The state information may be, for example, information determined based on information that can be acquired by a sensor or a device. Here, examples of the information that can be acquired by the sensor or the device include pedometer information, location information, and the like. In addition, the acquisition unit 11 may acquire only biometric information used for generation of an agitation determination model in the model generation unit 12 described later.

The non-patient biometric information may include biometric information of non-patients having different attributes. Here, the attribute is, for example, age group, age, gender, or the like. In other words, the non-patient biometric information may include, for example, biometric information of non-patients in different age groups. Here, the age group is divided according to an appropriately determined standard of age. As an example, the age group is divided by a ten-digit number for the age, and for example, a person of 20 years old or older and younger than 30 years old belongs to the twenties. Note that the attribute is not limited to this example as long as it is an attribute that may affect the biometric information.

The model generation unit 12 is a model generation means that generates an agitation determination model using patient biometric information and non-patient biometric information. Here, the agitation determination model is a model that determines whether the patient has become in agitation based on patient biometric information. A patient who is a subject of the agitation determination may be referred to as a target patient. The model generation unit 12 uses the biometric information acquired by the acquisition unit 11. The model generation unit 12 generates the agitation determination model using the patient biometric information and the non-patient biometric information as training data. The model generation unit 12 performs machine learning using the patient biometric information and the non-patient biometric information as training data, and generates an agitation determination model. Here, the training data includes training data related to an agitated state and training data related to a non-agitated state. The training data related to the agitated state is biometric information labeled as an agitated state. In addition, the training data related to the non-agitated state is biometric information labeled as a non-agitated state. The biometric information labeled as an agitated state may be referred to as a positive example, and the biometric information labeled as a non-agitated state may be referred to as a negative example.

As described above, the agitation determination model is a model that determines whether the patient has become in agitation based on patient biometric information. The agitation determination model outputs an agitation score using patient biometric information as an input. The agitation score is a value serving as an index indicating an agitated state or a non-agitated state. The agitation score is, for example, a value of zero or more and one or less. In this case, an agitation score closer to one indicates a higher possibility of being in an agitated state, and an agitation score closer to zero indicates a higher possibility of being in a non-agitated state. For example, a predetermined value of zero or more and one or less is used as a threshold, and an agitated state or a non-agitated state is determined based on the threshold. In addition, the agitation score may be a value expressed by a binary value of zero or one. In this case, the agitation score indicates one in an agitated state, and indicates zero in a non-agitated state.

The model generation unit 12 performs learning using, for example, a support vector machine (SVM), a neural network, other known machine learning methods, or the like, using biometric information labeled as an agitated state or a non-agitated state as training data.

Labeling of the agitated state or the non-agitated state with respect to the biometric information acquired by the acquisition unit 11 may be performed by the model generation unit 12 as described later, or may be performed by another device (not illustrated) or a user.

The training data related to the non-agitated state includes non-patient biometric information. In other words, the model generation unit 12 generates the agitation determination model by using the non-patient biometric information labeled as the non-agitated state.

The model generation unit 12 generates an agitation determination model using patient biometric information and non-patient biometric information in a resting state. In other words, the training data includes non-patient biometric information in a resting state. The resting state is, for example, a sleeping state, a relaxed state, or the like. Examples of the relaxed state include a state in which there is no physical or mental burden, a state in which the autonomic nerve is superior to the parasympathetic nerve, and the like. At this time, the model generation unit 12 uses the non-patient biometric information in a resting state as the training data of the negative example. In other words, the non-patient biometric information in a resting state is labeled as a non-agitated state.

Note that the resting state may be a state other than the non-resting state. The non-resting state includes at least one of a state of moving the body, a state of thinking, and a state of being stimulated. The resting state may be a state that is not the above state.

The training data related to the non-agitated state may include non-patient biometric information in a resting state. In other words, the model generation unit 12 generates the agitation determination model using the non-patient biometric information in a resting state labeled as a non-agitated state.

On the other hand, the training data related to the agitated state includes patient biometric information in an agitated state. In other words, the model generation unit 12 generates the agitation determination model using the patient biometric information in an agitated state labeled as the agitated state. As described above, the model generation unit 12 may use only the patient biometric information in an agitated state from pieces of patient biometric information. The training data related to the non-agitated state may include patient biometric information in a non-agitated state. In other words, the model generation unit 12 may generate the agitation determination model by using patient biometric information in a non-agitated state labeled as a non-agitated state.

Furthermore, the model generation unit 12 may have a configuration for determining the state of one or both of the patient and the non-patient related to the biometric information. At this time, the model generation unit 12 may label the biometric information as an agitated state or a non-agitated state based on the determined state. When the state information is acquired by the acquisition unit 11, the model generation unit 12 may determine one or both of the patient's agitated state or non-agitated state and the non-patient's resting state with reference to the state information at the time when the biometric information is measured.

As an example, the model generation unit 12 may determine whether the non-patient is in a resting state based on a piece of acquired biometric information or a combination of a plurality of pieces of acquired biometric information. In a case where it is determined that the non-patient is in a resting state based on a piece of the biometric information, the model generation unit 12 determines, for example, whether the non-patient is in a resting state based on information indicating the body movement of the non-patient. In this case, as an example, in a case where the value of the information indicating the body movement is lower than a predetermined value, the model generation unit 12 determines that the non-patient is in a resting state. Here, the information indicating the body movement includes acceleration acquired by the acceleration sensor. Furthermore, in a case where it is determined that the non-patient is in a resting state based on a combination of a plurality of pieces of biometric information, the model generation unit 12 determines that the non-patient is in a resting state based on, for example, a combination of results obtained by comparing the values of the plurality of pieces of biometric information with predetermined values. For example, in a case where the core body temperature is lower than a predetermined value such as normal body temperature of the non-patient and the respiratory rate is lower than the predetermined value, the model generation unit 12 determines that the patient is in a sleeping state, that is, a resting state.

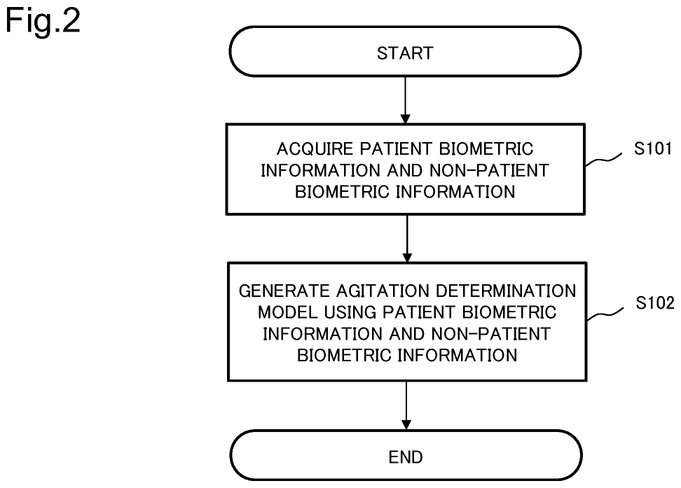

Next, an operation performed by the learning device 10 will be described with reference to FIG. 2. FIG. 2 is a flowchart illustrating an example of an operation performed by the learning device 10. The operation of the learning device 10 may be performed, for example, when a predetermined number or more of biometric information is accumulated in a storage device (not illustrated) or the like.

The acquisition unit 11 acquires patient biometric information and non-patient biometric information (step S101).

The model generation unit 12 generates an agitation determination model that determines whether the target patient has become in agitation based on the biometric information of the target patient by using the patient biometric information and the non-patient biometric information (step S102).

In the agitation determination model, learning is performed using training data in which whether the collected patient biometric information is in an agitated state is accurately labeled, which leads to improvement in accuracy.

However, the collected patient biometric information in the agitated state includes biometric information of patients in various states. As the patients in various states, for example, even when the patient is in an agitated state, it is not possible to confirm that the patient is in an agitated state from the behavior of the patient, and there is a possibility that a patient who looks at rest or the like is included. In this case, since the patient seems to be at rest, there is a possibility that the biometric information is labeled as a non-agitated state even though the patient is actually in an agitated state. In this manner, there is a possibility that the agitation determination model is generated using the training data in which the biometric information of the patient whose state is unclear is labeled. As a result, it may be difficult to generate a highly accurate agitation determination model.

In the learning device 10 according to the present example embodiment, the model generation unit 12 generates an agitation determination model using patient biometric information and non-patient biometric information. Regarding the non-patients, the correspondence between the state and the biometric information is less unclear compared to the case of the patients described above. In other words, there is a high possibility that the non-patient biometric information is labeled with high accuracy because it is easier to determine his/her state than the patient biometric information. Therefore, the learning device 10 can perform learning by adding the biometric information labeled with high accuracy to the training data. The learning device 10 thus can generate a highly accurate agitation determination model.

As an example, the learning device 10 generates an agitation determination model by using the patient biometric information as training data related to the agitated state and the non-patient biometric information as training data related to the non-agitated state in the model generation unit 12. By using such training data, a difference between the biometric information included in the training data related to the agitated state and the biometric information included in the training data related to the non-agitated state becomes clear. The learning device 10 thus can generate a highly accurate agitation determination model.

In the learning device 10 according to the present example embodiment, the model generation unit 12 generates an agitation determination model using patient biometric information, and non-patient biometric information in a resting state. As an example, a non-patient is a person whose chance of becoming in agitation is equal to or less than a predetermined probability while a patient is a person whose chance of becoming in agitation is equal to or more than the predetermined probability. Therefore, there is a high possibility that the resting state of the non-patient is the non-agitated state, and there is a high possibility that the non-patient biometric information in the resting state is accurately labeled as the non-agitated state. Therefore, the learning device 10 can perform learning by adding the biometric information accurately labeled as the non-agitated state to the training data. The learning device 10 thus can generate a highly accurate agitation determination model.

In the learning device 10 according to the present example embodiment, the model generation unit 12 generates an agitation determination model using patient biometric information in an agitated state and non-patient biometric information in a resting state. Since the patient biometric information in an agitated state is labeled as an agitated state after it is determined that the patient is actually in agitation, there is a high possibility that the biometric information is accurately labeled as the agitated state. In addition, as described above, there is a high possibility that the non-patient biometric information in a resting state is also accurately labeled as the non-agitated state. Therefore, the learning device 10 can perform learning using the biometric information labeled as the agitated state and the non-agitated state with high accuracy. The learning device 10 thus can generate a highly accurate agitation determination model.

In the present example embodiment, the non-patient biometric information can include biometric information of non-patients having different attributes. The biometric information may have different features related to differences in attributes. For example, heart rate variability, which is an example of biometric information, tends to decrease in accordance with an increase in age group or age. In the learning device 10 according to the present example embodiment, the model generation unit 12 generates the agitation determination model using the patient biometric information and the non-patient biometric information described above. With this configuration, the learning device 10 can widely learn the biometric information of the non-patients having a possibility of having different characteristics related to the difference in attributes of the patient or the non-patient and generate the agitation determination model. The learning device 10 thus can generate a highly accurate agitation determination model.

### Second Example Embodiment

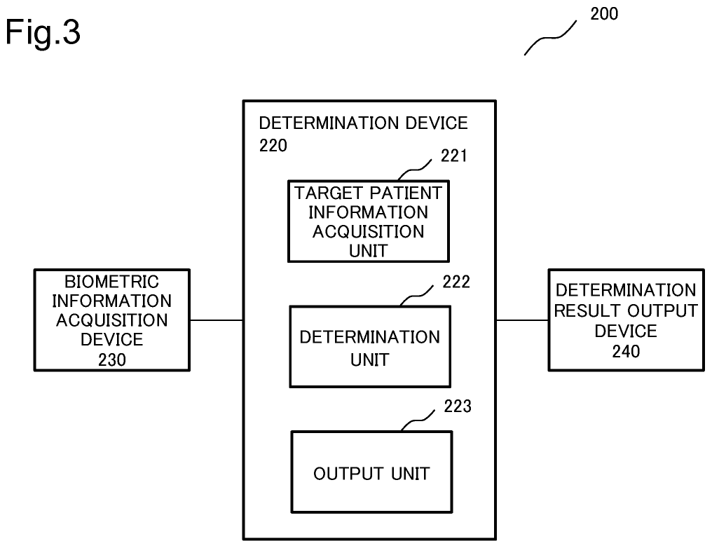

Hereinafter, a configuration of an agitation determination system 200 according to a second example embodiment will be described. FIG. 3 is a block diagram illustrating a configuration of the agitation determination system 200 according to the second example embodiment of the present invention. As illustrated in FIG. 3, the agitation determination system 200 according to the second example embodiment includes a determination device 220, a biometric information acquisition device 230, and a determination result output device 240. The determination device 220 and the biometric information acquisition device 230, and the determination device 220 and the determination result output device 240 are communicably connected to each other via a wireless or wired communication network such as Wi-Fi or Bluetooth (registered trademark).

The determination device 220 determines an agitated state of the target patient using the agitation determination model. The determination device 220 includes a target patient information acquisition unit 221, a determination unit 222, and an output unit 223. The determination device 220 is achieved, for example, in an information terminal such as a computer provided in a medical institution. The determination device 220 may be implemented on a cloud server, for example.

The target patient information acquisition unit 221 acquires biometric information of a target patient of agitated state determination. The target patient information acquisition unit 221 acquires the biometric information of the target patient by receiving the biometric information used in the agitated state determination of the target patient acquired by the biometric information acquisition device 230 to be described later.

The determination unit 222 is a determination means that determines whether the target patient has become in agitation using the biometric information of the target patient and the agitation determination model. Specifically, the determination unit 222 inputs the biometric information of the target patient to the agitation determination model to obtain an agitation score. Then, the determination unit 222 determines an agitated state or a non-agitated state of the target patient based on the agitation score.

The agitation determination model is a model generated by the learning device 10 in the first example embodiment. In other words, the agitation determination model according to the present example embodiment is a trained model generated in advance using the patient biometric information and the non-patient biometric information. The determination unit 222 acquires the agitation determination model stored in a storage device (not illustrated) or the like, and determines an agitated state of the target patient.

The output unit 223 outputs the agitated state determination result of the target patient by the determination unit 222. The output unit 223 outputs the determination result to the determination result output device 240 described later. The output unit 223 outputs the determination result in a format that can be output by the determination result output device 240. For example, in a case where the determination result output device 240 includes a display unit such as a display that outputs a determination result, the output unit 223 includes a function as a display control unit that controls the display unit. In this manner, the output unit 223 functions as a unit that controls the determination result output device 240 according to the format of the determination result output in the determination result output device 240.

The biometric information acquisition device 230 is a device that acquires biometric information of a patient. The biometric information acquisition device 230 is, for example, a wearable device or the like. The biometric information acquisition device 230 is, for example, a device including at least one sensor that acquires biometric information of a patient by being worn on the patient. The biometric information and the sensor are as described above. Furthermore, the biometric information acquisition device 230 may be, for example, an imaging device installed in a patient's room or a device that acquires one or both of patient's voice and sound information in a surrounding environment of the patient. In this case, the biometric information acquisition device 230 performs processing of extracting the biometric information of the patient based on the acquired image information and sound information.

The determination result output device 240 outputs the determination result of the agitated state of the target patient acquired from the determination device 220. The determination result output device 240 is, for example, an information terminal such as a computer provided in a medical institution. The determination result output device 240 may be an information terminal such as a tablet terminal or a smartphone used by a medical worker. The determination result output device 240 includes, for example, at least one of a display unit capable of displaying characters and images such as a display, a sound output unit capable of outputting sound such as a speaker, and the like. The determination result output device 240 presents the determination result of the agitated state of the target patient to the medical worker using at least one of the display means, the sound output means, and the like.

The determination result output device 240 may output the biometric information of the target patient acquired by the biometric information acquisition device 230 together with the determination result of the agitated state of the target patient. In this case, the biometric information acquisition device 230 and the determination result output device 240 are connected so as to be able to communicate with each other via a wireless or wired communication network as described above, for example.

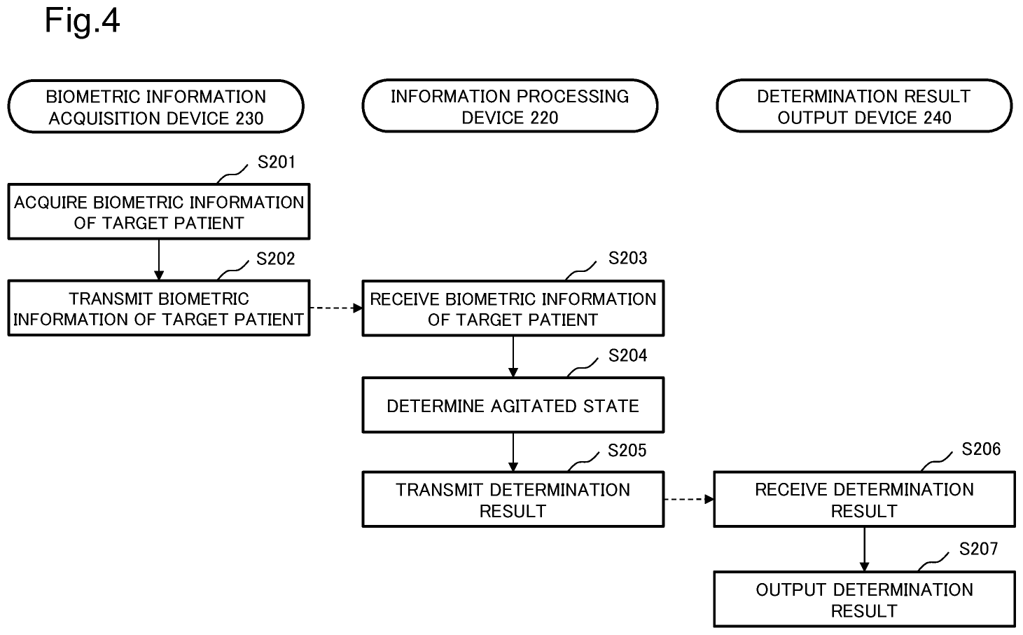

An operation performed by the agitation determination system 200 will be described with reference to FIG. 4. FIG. 4 is a flowchart illustrating an example of an operation performed by the agitation determination system 200.

The biometric information acquisition device 230 acquires biometric information of the target patient (step S201). Then, the biometric information acquisition device 230 transmits the acquired biometric information of the target patient to the determination device 220 (step S202).

The target patient information acquisition unit 221 of the determination device 220 receives the biometric information of the target patient from the biometric information acquisition device 230 (step S203). The determination unit 222 determines an agitated state of the target patient using the biometric information of the target patient and the agitation determination model (step S204). The output unit 223 transmits the determination result of the agitated state of the target patient by the determination unit 222 to the determination result output device 240 (step S205).

The determination result output device 240 receives the determination result of the agitated state of the target patient from the determination device 220 (step S206). Then, the determination result output device 240 outputs the determination result of the agitated state of the target patient to the medical worker or the like using at least one of the display means, the sound output means, and the like (step S207).

The agitation determination system 200 in the present example embodiment determines an agitated state of the target patient by using biometric information of the target patient and an agitation determination model in the determination device 220. The agitation determination model is a model generated by the learning device 10 according to the first example embodiment. The determination device 220 can accurately determine the agitated state of the target patient by using the agitation determination model. The determination result in which the agitated state of the target patient is accurately determined is output to the determination result output device 240, whereby the medical worker or the like can efficiently grasp the agitated state of the patient. In this manner, the agitation determination system 200 contributes to improvement of work efficiency of medical workers and the like.

The agitation determination system 200 may include the learning device 10 according to the first example embodiment. In other words, the agitation determination system 200 may be a system including a learning device. In this case, the determination device 220 determines the agitated state of the target patient using the agitation determination model generated by the learning device 10. The agitation determination system 200 may have a relearning function. The learning device 10 further uses biometric information of the target patient acquired by the target patient information acquisition unit 221 and non-patient biometric information acquired from a storage device or the like (not illustrated), so that the agitation determination system 200 generates a retrained agitation determination model. The agitation determination system 200 may perform relearning when the determination result of the agitated state of the target patient output by the determination device 220 does not reach predetermined accuracy. The determination device 220 in the agitation determination system 200 may include a configuration included in the learning device 10.

### Hardware Configuration for Achieving Each Component of Example Embodiment

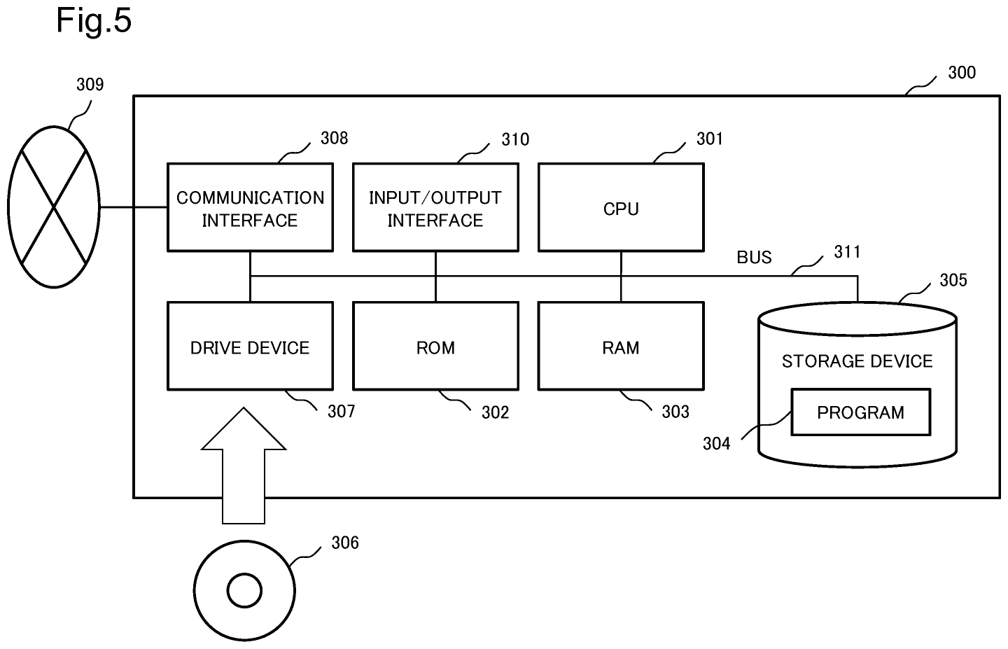

In each example embodiment of the present invention, each component of each device and system represents a block of functional units. A part or all of each component of each device and system is achieved by, for example, an arbitrary combination of an information processing device 300 and a program as illustrated in FIG. 5. The information processing device 300 includes the following configuration as an example.
central processing unit (CPU) 301
read only memory (ROM) 302
random access memory (RAM) 303
program 304 loaded into RAM 303
storage device 305 storing program 304
drive device 307 that reads and writes in recording medium 306
communication interface 308 connected with communication network 309
input/output interface 310 for inputting and outputting data
bus 311 that connects components

Each component of each device in each example embodiment is achieved by the CPU 301 acquiring and executing the program 304 for achieving these functions. The program 304 for achieving the function of each component of each device is stored in the storage device 305 or the RAM 303 in advance, for example, and is read by the CPU 301 as necessary. Note that the program 304 may be supplied to the CPU 301 via the communication network 309, or may be stored in advance in the recording medium 306, and the drive device 307 may read the program and supply the program to the CPU 301.

There are various modifications of the implementation method of each device. For example, each device may be achieved by an arbitrary combination of the information processing device 300 and the program for each component. A plurality of components included in each device may be achieved by an arbitrary combination of one information processing device 300 and a program.

A part or all of each component of each device is achieved by a general-purpose or dedicated circuit including a processor or the like, or a combination thereof. These may be configured by a single chip or may be configured by a plurality of chips connected via a bus. Some or all of the components of each device may be achieved by a combination of the above-described circuit and the like and a program.

In a case where some or all of the components of each device are achieved by a plurality of information processing devices, circuits, and the like, the plurality of information processing devices, circuits, and the like may be arranged in a centralized manner or in a distributed manner. For example, the information processing device, the circuit, and the like may be achieved in a manner of being connected to one another via a communication network, such as a client and server system or a cloud computing system.

In the above description, examples of generating a model for determining an agitated state of a patient has been described. However, the present invention is not limited to the model for determining an agitated state, and can be applied to any scene in which a model for determining a state of a subject such as a patient is generated.

While the present invention has been particularly shown and described with reference to exemplary embodiments thereof, the invention is not limited to these example embodiments. It will be understood by those of ordinary skill in the art that various changes in form and details may be made therein without departing from the spirit and scope of the present invention as defined by the claims. In addition, the configurations of the above-described example embodiments may be combined or some components may be interchanged.

Some or all of the above example embodiments can also be described as the following supplementary notes, but are not limited to the following.

### (Supplementary Note 1)

A learning device comprising:
an acquisition means configured to acquire patient biometric information and non-patient biometric information, the patient being a person who may possibly become in agitation; and
a model generation means configured to generate an agitation determination model that determines whether a target patient has become in agitation or has not become in agitation based on biometric information of the target patient by using the patient biometric information and the non-patient biometric information.

### (Supplementary Note 2)

The learning device according to supplementary note 1, wherein the model generation means generates the agitation determination model using the patient biometric information and the non-patient biometric information as training data.

### (Supplementary Note 3)

The learning device according to supplementary note 2, wherein the training data includes non-patient biometric information in a resting state.

### (Supplementary Note 4)

The learning device according to supplementary note 2 or 3, wherein the training data related to the non-agitated state includes the non-patient biometric information.

### (Supplementary Note 5)

The learning device according to supplementary note 3 or 4, wherein the resting state of the non-patient includes a sleeping state.

### (Supplementary Note 6)

The learning device according to any one of supplementary notes 2 to 5, wherein the training data related to the agitated state includes the patient biometric information in an agitated state.

### (Supplementary Note 7)

The learning device according to any one of supplementary notes 2 to 6, wherein the training data related to the non-agitated state includes the patient biometric information in a non-agitated state.

### (Supplementary Note 8)

The learning device according to any one of supplementary notes 1 to 7, wherein the non-patient is a person whose chance of becoming in agitation is equal to or less than a predetermined probability.

### (Supplementary Note 9)

The learning device according to any one of supplementary notes 1 to 8, wherein the non-patient is at least one of a person who can perform daily life activities by himself/herself, a person who does not have an underlying disease, and a person who does not need assistance or care of another person.

### (Supplementary Note 10)

The learning device according to any one of supplementary notes 1 to 9, wherein the non-patient biometric information includes biometric information of non-patients in different age groups.

### (Supplementary Note 11)

A determination device comprising
a determination means configured to determine whether the target patient has become in agitation using the biometric information of the target patient and the agitation determination model,
wherein the agitation determination model is a trained model generated by the learning device according to any one of supplementary notes 1 to 10.

### (Supplementary Note 12)

A method for generating a trained model, the method causing a computer to perform a process comprising:
acquiring patient biometric information and non-patient biometric information, the patient being a person who may become in agitation; and
generating an agitation determination model for determining whether a target patient has become in agitation based on biometric information of the target patient by using the patient biometric information and the non-patient biometric information.

### (Supplementary Note 13)

A recording medium storing a program that causes a computer to perform a process comprising:
acquiring patient biometric information and non-patient biometric information, the patient being a person who may become in agitation; and
generating an agitation determination model for determining whether a target patient has become in agitation based on biometric information of the target patient by using the patient biometric information and the non-patient biometric information.

### REFERENCE SIGNS LIST

10 learning device
11 acquisition unit
12 model generation unit
200 agitation determination system
220 determination device
221 target patient information acquisition unit
222 determination unit
223 output unit
230 biometric information acquisition device
240 determination result output device
300 Information processing device
301 CPU
302 ROM
303 RAM
304 program
305 storage device
306 recording medium
307 drive device
308 communication interface
309 communication network
310 input/output interface
311 bus

What is claimed is:

1. A learning device comprising:
a memory storing instructions; and
at least one processor configured to execute the instructions to:
acquire patient biometric information and non-patient biometric information, the patient being a person who may possibly become in agitation; and
generate an agitation determination model that determines whether a target patient has become in agitation or has not become in agitation based on biometric information of the target patient by using the patient biometric information and the non-patient biometric information.

2. The learning device according to claim 1, wherein
the at least one processor is further configured to execute the instructions to:
generate the agitation determination model using the patient biometric information and the non-patient biometric information as training data.

3. The learning device according to claim 2, wherein the training data includes non-patient biometric information in a resting state.

4. The learning device according to claim 2, wherein the training data related to the non-agitated state includes the non-patient biometric information.

5. The learning device according to claim 3, wherein the resting state of the non-patient includes a sleeping state.

6. The learning device according to claim 2, wherein the training data related to the agitated state includes the patient biometric information in an agitated state.

7. The learning device according to claim 2, wherein the training data related to the non-agitated state includes the patient biometric information in a non-agitated state.

8. The learning device according to claim 1, wherein the non-patient is a person whose chance of becoming agitated is equal to or less than a predetermined probability.

9. The learning device according to claim 1, wherein the non-patient is at least one of a person who can perform daily life activities by himself/herself, a person who does not have an underlying disease, and a person who does not need assistance or care of another person.

10. The learning device according to claim 1, wherein the non-patient biometric information includes biometric information of non-patients in different age groups.

11. A determination device comprising
determine whether the target patient has become in agitation using the biometric information of the target patient and the agitation determination model,
wherein the agitation determination model is a trained model generated by the learning device according to claim 1.

12. A method for generating a trained model by a computer, comprising:
acquiring patient biometric information and non-patient biometric information, the patient being a person who may become agitation; and
generating an agitation determination model for determining whether a target patient has become in agitation based on biometric information of the target patient by using the patient biometric information and the non-patient biometric information.

13. A recording medium non-transitorily storing a program that causes a computer to perform a process comprising:
acquiring patient biometric information and non-patient biometric information, the patient being a person who may become in agitation; and
generating an agitation determination model for determining whether a target patient has become agitated based on biometric information of the target patient by using the patient biometric information and the non-patient biometric information.
